(19) United States
(12) Patent Application Publication
(10) Pub. No.: US 2019/0161547 A1
(43) Pub. Date: May 30, 2019

(54) METHODS FOR REGULATING ENDOGENOUS PRODUCTION OF CHECKPOINT MOLECULE ANTAGONISTS

(71) Applicant: Wyvern Pharmaceutical Inc., Calgary (CA)

(72) Inventor: Bradley G. THOMPSON, Calgary (CA)

(73) Assignee: Wyvern Pharmaceutical Inc.

(21) Appl. No.: 15/826,267

(22) Filed: Nov. 29, 2017

(51) Int. Cl. C07K 16/28; A61K 45/06; A61P 35/00

(52) U.S. Cl. CPC C07K 16/2818; C07K 16/2827; A61K 2039/507; A61P 35/00; A61K 45/06

(57) ABSTRACT

The present disclosure relates to one or more agents, therapies, treatments, and methods of use of the agents and/or therapies and/or treatments for decreasing production and/or functionality of one or more immune checkpoint molecules. Embodiments of the present disclosure can be used as a therapy or a treatment for a subject that has an infection or infections, or cancer or cancers

### TECHNICAL FIELD

The present disclosure generally relates to molecules that participate within one or more cellular-signaling cascades. In particular, the present disclosure relates to agents, therapies, and methods of use of compounds, agents and/or therapies for increasing production of one or more antagonists of checkpoint molecules as a therapy or treatment of conditions resulting from an infection and/or cancer.

### BACKGROUND

The immune system has evolved to allow an individual host to differentiate between self and foreign matter. A number of cascades of signaling molecules and immune cells are well characterized for their ability to recognize foreign matter and for calling upon the production and stimulation of effector cells of the immune system to kill, break down, consume, or sheath the foreign matter in order to protect the host.

It is known that under various conditions the immune system will not be effective in recognizing foreign matter or addressing the risk associated with foreign matter.

Immune checkpoint molecules, in particular checkpoint proteins (CPPs), are known participants in the immune system's responses to foreign matter. CPPs can be categorized as stimulatory or inhibitory. Many stimulatory CPPs have been identified as participating, either directly or indirectly, in increasing a host's immune response or protecting cells from the host's immune response. Many inhibitory CPPs have also been identified that function to participate, either directly or indirectly, in decreasing the host's immune response. For example, it is known that cells with chronic viral infections have increased levels of CPPs on the cells' surface, which protects the cells from the host's immune response. The exogenous addition of a CPP antagonist to cell surfaces may have therapeutic benefit in conditions were the cell surfaces have increased levels of one or more CPPs.

Known approaches for the treatment of cancer where the levels of CPPs on cell surfaces are elevated include commercially available pharmaceutical products that target specific CPPs. For example, pembrolizumab is a targeted PD-1 antagonist, atezolizumab is a targeted PD-L1 antagonist, durvalumab is a targeted PD-L1 antagonist, avelumab is a targeted PD-L1 antagonist, nivolumab is a targeted PD-1 inhibitor and ipilumumab is a targeted CTLA-4 antagonist. These pharmaceutical products are each comprised of antibodies against the targeted CPP and when added exogenously these pharmaceutical products can each act as an antagonist to the targeted CPPs.

Another known approach to the treatment of cancer where the levels of CPPs on cell surfaces are elevated, is to create a viral vector that includes one or more genes that cause the production of an anti-CPP antibody. For example, Newcastle's disease virus (NDV) has been engineered to include one or more genes that cause the production of an antibody against CTLA-4.

Another example of a checkpoint molecule that participates in the immune system's response to foreign matter is phosphatidylserine (PS). PS is an inhibitory checkpoint molecule that functions to participate, either directly or indirectly, in decreasing the host's immune response. A known approach to the treatment of cancer where the levels of PS on cell surfaces are elevated, is to treat exogenously with annexin V, which is a naturally occurring protein antagonist to PS.

### SUMMARY

Some embodiments of the present disclosure relate to a method of making an agent/target cell complex, the method comprises a step of administering a therapeutically effective amount of the agent to a subject. Wherein in some embodiments of the present disclosure the agent/target cell complex causes the endogenous production of one or more antagonists of one or more checkpoint molecules, such as one or more checkpoint proteins (CPPs) and/or phosphatidylserine (PS). Wherein in some embodiments of the present disclosure the agent/target cell complex causes the endogenous production of one or more regulatory molecules that inhibit the production and/or functionality of one or more checkpoint molecules. Wherein in some embodiments of the present disclosure the agent/target cell complex causes the endogenous production of both of the one or more antagonists and the one or more regulatory molecules so that the production and/or functionality of one or more checkpoint molecules are inhibited.

Some embodiments of the present disclosure relate to a method of making an agent/target cell complex. The method comprises a step of administering a therapeutically effective amount of an agent to a subject, wherein the agent/target cell complex increases the subject's endogenous production and/or a functionality of an antagonist of a checkpoint molecule.

Some embodiments of the present disclosure relate to a pharmaceutical composition that comprises an agent, a pharmaceutically acceptable carrier and/or an excipient. The agent decreases a production and/or a functionality of one or more checkpoint molecules and/or the agent increases a production and/or a functionality of one or more regulatory molecules that decrease a production and/or a functionality of the one or more checkpoint molecules.

Some embodiments of the present disclosure relate to a method of treating an infection. The method comprises a step of administering to a subject a therapeutically effective amount of an agent that decreases a production and/or a functionality of three or more checkpoint molecules.

Some embodiments of the present disclosure relate to a method of treating a cancer. The method comprises a step of administering to a subject a therapeutically effective amount of an agent that decreases a production and/or a functionality of three or more checkpoint molecules.

In some embodiments of the present disclosure at least two agents may be administered to form at least two different types of agent/target cell complexes and each type of agent/target cell complex inhibits the production and/or functionality of at least one checkpoint molecule.

In some embodiments of the present disclosure at least three agents may be administered to form at least three different types of agent/target cell complexes and each type of agent/target cell complex inhibits the production and/or functionality of at least one checkpoint molecule.

In some embodiments of the present disclosure at least four agents may be administered to form at least four different types of agent/target cell complexes and each type of agent/target cell complex inhibits the production and/or functionality of at least one checkpoint molecule.

In some embodiments of the present disclosure at least five agents may be administered to form at least five different types of agent/target cell complexes and each type of agent/target cell complex inhibits the production and/or functionality of at least one checkpoint molecule.

In some embodiments of the present disclosure at least six agents may be administered to form at least six different types of agent/target cell complexes and each type of agent/target cell complex inhibits the production and/or functionality of at least one checkpoint molecule.

Some embodiments of the present disclosure relate to a method of making an agent/target cell complex, the method comprising a step of administering a sufficient amount of an agent to a target cell whereby the agent/target cell complex is formed. Wherein in some embodiments of the present disclosure the agent/target cell complex causes the endogenous production of one or more antagonists of one or more checkpoint molecules, for example one or more CPPs and/or PS. Wherein in some embodiments of the present disclosure the agent/target cell complex causes the endogenous production of one or more regulatory molecules that inhibit the production and/or functionality of one or more checkpoint molecules. Wherein in some embodiments of the present disclosure the agent/target cell complex causes the endogenous production of both of the one or more antagonists and the one or more regulatory molecules so that the production and/or functionality of one or more checkpoint molecules are inhibited.

Some embodiments of the present disclosure relate to a pharmaceutical composition that comprises an agent, a pharmaceutically acceptable carrier, and/or an excipient. Wherein in some embodiments of the present disclosure the agent causes the endogenous production of one or more antagonists of one or more checkpoint molecules, for example one or more checkpoint molecules, for example one or more CPPs and/or PS. Wherein in some embodiments of the present disclosure the agent causes the endogenous production of one or more regulatory molecules that inhibit the production and/or functionality of one or more checkpoint molecules. Wherein in some embodiments of the present disclosure the agent causes the endogenous production of both of the one or more antagonists and the one or more regulatory molecules so that the production and/or functionality of one or more checkpoint molecules are inhibited.

Some embodiments of the present disclosure relate to a kit used for treatment of a condition or for delivery of a therapy to a subject. The kit comprises a unit dosage of an agent, a carrier for the unit dosage, and instructions for administering the unit dosage to the subject. Wherein in some embodiments of the present disclosure the agent causes the endogenous production of one or more antagonists of one or more checkpoint molecules, for example one or more CPPs and/or PS. Wherein in some embodiments of the present disclosure the agent causes the endogenous production of one or more regulatory molecules that inhibit the production and/or functionality of one or more checkpoint molecules. Wherein in some embodiments of the present disclosure the agent causes the endogenous production of both of the one or more antagonists and the one or more regulatory molecules so that the production and/or functionality of one or more checkpoint molecules are inhibited. The carrier may be a solid carrier, such as a pill or tablet, or a liquid. The instructions may describe how the solid carrier may be administered to a subject for an optimal effect. The instructions may also describe how the liquid carrier may be administered to a subject by various routes of administration.

Some embodiments of the present disclosure relate to a method of treating a condition. The method comprises a step of administering to a subject a therapeutically effective amount of an agent that may cause one, or some or all of: the endogenous production of one or more antagonists of one or more checkpoint molecules, for example one or more CPPs and/or PS; or the endogenous production of one or more regulatory molecules that inhibits the production or functionality of one or more checkpoint molecules.

Some embodiments of the present disclosure relate to a method for causing the endogenous production of one, or some or all of: an antagonist of one or more checkpoint molecules, for example one or more CPPs and/or PS; or a regulatory molecule that inhibits the production or functionality of one or more checkpoint molecules, the method comprising a step of making an agent/target cell complex.

Embodiments of the present disclosure relate to at least one approach for inducing or increasing the endogenous production of one, or some or all of: one or more antagonists of one or more checkpoint molecules, for example CPPs and/or PS; or one or more regulatory molecules that inhibit the production or functionality of one or more checkpoint molecules. A first approach utilizes one or more gene vectors that contain nucleotide sequences and/or genes that cause the recipient of the one or more gene vectors to produce, or increase production of, one or more antagonists to checkpoint molecules: CTLA-4, PD-1, PD-L1, PD-L2, IDO1 and other non-protein checkpoint molecules, such as PS. Another approach utilizes one or more gene vectors that contain nucleotide sequences and/or genes that cause the recipient of the one or more gene vectors to produce, or increase production of, one or more regulatory molecules that inhibit the production or functionality of one or more checkpoint molecules. Another approach utilizes one or more gene vectors that contain nucleotide sequences and/or genes that cause the recipient of the one or more gene vectors to produce, or increase production of, the one or more antagonists and the one or more regulatory molecules.

Without being bound by any particular theory, therapies or treatments that comprise the use of exogenous antagonists of a single checkpoint molecule, or the use of viral vectors that cause the production of an antibody of a single checkpoint molecule, may be limited by the effectiveness of the treatment or therapy to access a subject's affected cells. Furthermore, the exogenous antagonists' impact only one of many checkpoint molecules that may be found on the target cells' surface, which means that other checkpoint molecules may still influence the impact of the host's immune response.

An agent that induces endogenous production of one, or some or all of: one or more antagonists of one or more checkpoint molecules, for example one or more CPPs and/or PS; or one or more regulatory molecules that inhibit the production and/or functionality of one or more checkpoint molecules, may be more effective at unmasking the target cells from the immune system than an exogenously added, single checkpoint molecule antagonist or a single anti-CPP antibody that is coded for by a vector.

Embodiments of the present disclosure may be useful for treating conditions where the subject's immune system is not effective in recognizing foreign matter, for example during an infection, infections, cancer or cancers. Some examples of infections include, but are not limited to: a chronic viral infection, a bacterial infection, a mycoplasma infection, a plasmodium infection, amoeba, fungus, parasite infection.

### DETAILED DESCRIPTION

### Definitions

Unless defined otherwise, all technical and scientific terms used herein have the meanings that would be commonly understood by one of skill in the art in the context of the present description. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

As used herein, the singular forms “a”, “an”, and “the” include plural references unless the context clearly dictates otherwise. For example, reference to “an agent” includes one or more agents and reference to “a subject” or “the subject” includes one or more subjects.

As used herein, the terms “about” or “approximately” refer to within about 25%, preferably within about 20%, preferably within about 15%, preferably within about 10%, preferably within about 5% of a given value or range. It is understood that such a variation is always included in any given value provided herein, whether or not it is specifically referred to.

As used herein, the term “agent” refers to a substance that, when administered to a subject, causes one or more chemical reactions and/or one or more physical reactions and/or or one or more physiological reactions and/or one or more immunological reactions in the subject.

As used herein, the term “antagonist” refers to an agent that can, directly or indirectly, inhibit a physiologic activity and/or production of a target molecule within a subject that receives the agent.

As used herein, the term “ameliorate” refers to improve and/or to make better and/or to make more satisfactory.

As used herein, the term “biomolecule” refers to a carbohydrate, a protein, an amino acid sequence, a nucleic acid, a lipid, a primary metabolite, a secondary metabolite that is found within a subject. A biomolecule may be endogenous or exogenous.

As used herein, the term “cell” refers to a single cell as well as a plurality of cells or a population of the same cell type or different cell types. Administering an agent to a cell includes in vivo, in vitro and ex vivo administrations or combinations thereof.

As used herein, the term “complex” refers to an association, either direct or indirect, between one or more particles of an agent and one or more target cells. This association results in a change in the metabolism of the target cell. As used herein, the phrase “change in metabolism” refers to an increase or a decrease in the one or more target cells' production of deoxyribonucleic acid (DNA), ribonucleic acid (RNA), one or more proteins, or any post-translational modifications of one or more proteins.

As used herein, the term “effector molecule” refers to a molecule within a subject that can directly or indirectly regulate the metabolic activity of a target cell by increasing or decreasing the production of DNA, RNA, amino-acid sequences and/or by increasing or decreasing any post-translational modifications of one or more proteins.

As used herein, the term “endogenous” refers to the synthesis, production and/or modification of a molecule that originates within a subject.

As used herein, the term “excipient” refers to any substance, not itself an agent, which may be used as a component within a pharmaceutical composition or a medicament for administration of a therapeutically effective amount of the agent to a subject. Additionally or alternatively an excipient may alone, or in combination with further chemical components, improve the handling and/or storage properties and/or to permit or facilitate formation of a dose unit of the agent. Excipients include, but are not limited to, one or more of: a binder, a disintegrant, a diluent, a buffer, a taste enhancer, a solvent, a thickening agent, a gelling agent, a penetration enhancer, a solubilizing agent, a wetting agent, an antioxidant, a preservative, a surface active agent, a lubricant, an emollient, a substance that is added to mask or counteract a disagreeable odor, fragrances or taste, a substance added to improve appearance or texture of the composition and a substance used to form the pharmaceutical compositions or medicaments. Any such excipients can be used in any dosage forms according to the present disclosure. The foregoing classes of excipients are not meant to be exhaustive but are provided merely as illustrative of what a person of skill in the art would know and would also recognize that additional types and combinations of excipients may be used to achieve delivery of a therapeutically effective amount of the agent to a subject through one or more routes of administration.

As used herein, the term “exogenous” refers to a molecule that is within a subject but that did not originate within the subject.

As used herein, the terms “inhibit”, “inhibiting”, and “inhibition” refer to a decrease in activity, response, or other biological parameter of a biologic process, disease, disorder or symptom thereof. This can include but is not limited to the complete ablation of the activity, response, condition, or disease. This may also include, for example, a 10% reduction in the activity, response, condition, or disease as compared to the native or control level. Thus, the reduction can be a 10%, 20%, 30%, 40%, 50%, 60%, 70%, 80%, 90%; 100%, or any amount of reduction in between the specifically recited percentages, as compared to native or control levels.

As used herein, the term “subject” refers to any therapeutic target that receives the agent. The subject can be a vertebrate, for example, a mammal including a human. The term “subject” does not denote a particular age or sex. The term “subject” also refers to one or more cells of an organism, an in vitro culture of one or more tissue types, an in vitro culture of one or more cell types; ex vivo preparations; and a sample of biological materials such as tissue and/or biological fluids.

As used herein, the term “medicament” refers to a medicine and/or pharmaceutical composition that comprises the agent and that can promote recovery from a disease, disorder or symptom thereof and/or that can prevent a disease, disorder or symptom thereof and/or that can inhibit the progression of a disease, disorder, or symptom thereof.

As used herein, the term “patient” refers to a subject that is afflicted with an infectious disease. The term “patient” includes human and veterinary subjects.

As used herein, the term “pharmaceutical composition” means any composition for administration of an agent to a subject in need of therapy or treatment of a disease, disorder or symptom thereof. Pharmaceutical compositions may include additives such as pharmaceutically acceptable carriers, pharmaceutically accepted salts, excipients and the like. Pharmaceutical compositions may also additionally include one or more further active-ingredients such as antimicrobial agents, anti-inflammatory agents, anaesthetics, analgesics, and the like.

As used herein, the term “pharmaceutically acceptable carrier” refers to an essentially chemically inert and nontoxic component within a pharmaceutical composition or medicament that does not inhibit the effectiveness and/or safety of the agent. Some examples of pharmaceutically acceptable carriers and their formulations are described in Remington (1995, The Science and Practice of Pharmacy (19th ed.) ed. A. R. Gennaro, Mack Publishing Company, Easton, Pa.), the disclosure of which is incorporated herein by reference. Typically, an appropriate amount of a pharmaceutically acceptable carrier is used in the formulation to render the formulation isotonic. Examples of suitable pharmaceutically acceptable carriers include, but are not limited to: saline solutions, glycerol solutions, ethanol, N-(1(2,3-dioleyloxy)propyl)-N,N,N-trimethylammonium chloride (DOTMA), diolesylphosphotidylethanolamine (DOPE), and liposomes. Such pharmaceutical compositions contain a therapeutically effective amount of the agent, together with a suitable amount of one or more pharmaceutically acceptable carriers and/or excipients so as to provide a form suitable for proper administration to the subject. The formulation should suit the route of administration. For example, oral administration may require enteric coatings to protect the agent from degrading within portions of the subject's gastrointestinal tract. In another example, injectable routes of administration may be administered in a liposomal formulation to facilitate transport throughout a subject's vascular system and to facilitate delivery across cell membranes of targeted intracellular sites.

As used herein, the phrases “prevention of” and “preventing” refer to avoiding an onset or progression of a disease, disorder, or a symptom thereof.

As used herein, the terms “production”, “producing” and “produce” refer to the synthesis and/or replication of DNA, the transcription of one or more sequences of RNA, the translation of one or more amino acid sequences, the post-translational modifications of amino acid sequences, the synthesis or altered functionality of one or more regulatory molecules that can influence the production or functionality of an effector molecule or an effector cell.

As used herein, the terms “promote”, “promotion”, and “promoting” refer to an increase in an activity, response, condition, disease process, or other biological parameter. This can include but is not limited to the initiation of the activity, response, condition, or disease process. This may also include, for example, a 10% increase in the activity, response, condition, or disease as compared to the native or control level. Thus, the increase in an activity, response, condition, disease, or other biological parameter can be a 10%, 20%, 30%, 40%, 50%, 60%, 70%, 80%, 90%, 100%, or more, including any amount of increase in between the specifically recited percentages, as compared to native or control levels.

As used herein, the term “prophylactic administration” refers to the administration of any composition to a subject, in the absence of any symptom or indication of a disease or disorder, to prevent the occurrence of and/or the progression of the disease or disorder within the subject.

As used herein, the terms “signal molecule”, “signalling molecule” and “regulatory molecule” can be used interchangeably and they refer to a molecule that can directly or indirectly affect the production and/or functionality of an effector molecule or effector cell. Signal molecules can be biomolecules and they can act as a direct ligand on a target cell or they may influence the levels or functionality of a downstream ligand.

As used herein, the term “target cell” refers to one or more cells and/or cell types that are deleteriously affected, either directly or indirectly, by a disease.

As used herein, the terms “treat”, “treatment” and “treating” refer to obtaining a desired pharmacologic and/or physiologic effect. The effect may be prophylactic in terms of completely or partially preventing an occurrence of a disease, disorder or symptom thereof and/or the effect may be therapeutic in providing a partial or complete amelioration or inhibition of a disease, disorder, or symptom thereof. Additionally, the term “treatment”, refers to any treatment of a disease, disorder, or symptom thereof in a subject and includes: (a) preventing the disease from occurring in a subject which may be predisposed to the disease but has not yet been diagnosed as having it; (b) inhibiting the disease; and (c) ameliorating the disease.

As used herein, the term “therapeutically effective amount” refers to the amount of the agent used that is of sufficient quantity to ameliorate, treat and/or inhibit one or more of a disease, disorder or a symptom thereof. The “therapeutically effective amount” will vary depending on the agent used, the route of administration of the agent and the severity of the disease, disorder or symptom thereof. The subject's age, weight and genetic make-up may also influence the amount of the agent that will be a therapeutically effective amount.

As used herein, the terms “unit dosage form” and “unit dose” refer to a physically discrete unit that is suitable as a unitary dose for patients. Each unit contains a predetermined quantity of the agent and optionally, one or more suitable pharmaceutically acceptable carriers, one or more excipients, one or more further active-ingredients, or combinations thereof. The amount of agent within each unit is a therapeutically effective amount.

In one embodiment of the present disclosure, the pharmaceutical compositions disclosed herein comprise an agent as described above in a total amount by weight of the composition of about 0.1% to about 95%. For example, the amount of the agent by weight of the pharmaceutical composition may be about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%>, about 2.2%, about 2.3%, about 2.4%, about 2.5%, about 2.6%, about 2.7%, about 2.8%, about 2.9%, about 3%, about 3.1%, about 3.2%, about 3.3%, about 3.4%, about 3.5%, about 3.6%, about 3.7%, about 3.8%, about 3.9%, about 4%, about 4.1%, about 4.2%, about 4.3%, about 4.4%, about 4.5%, about 4.6%, about 4.7%, about 4.8%. about 4.9%, about 5%, about 5.1%, about 5.2%, about 5.3%, about 5.4%, about 5.5%, about 5.6%, about 5.7%, about 5.8%, about 5.9%, about 6%, about 6.1%, about 6.2%, about 6.3%, about 6.4%, about 6.5%, about 6.6%, about 6.7%, about 6.8%, about 6.9%, about 7%, about 7.1%, about 7.2%, about 7.3%, about 7.4%, about 7.5%, about 7.6%, about 7.7%, about 7.8%, about 7.9%, about 8%, about 8.1%, about 8.2%, about 8.3%, about 8.4%, about 8.5%, about 8.6%, about 8.7%, about 8.8%, about 8.9%, about 9%, about 9.1%, about 9.2%, about 9.3%, about 9.4%, about 9.5%, about 9.6%, about 9.7%, about 9.8%, about 9.9%, about 10%, about 11%, about 12%, about 13%, about 14%, about 15%, about 16%, about 17%, about 18%, about 19%, about 20%, about 25%, about 30%, about 35%, about 40%, about 45%, about 50%, about 55%, about 60%, about 65%, about 70%, about 75%, about 80%, about 85%, about 90% or about 95%.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also, encompassed within the disclosure, subject to any specifically excluded limit in the stated range. Where the stated range includes one or both of the limits, ranges excluding either or both of those included limits are also included in the disclosure.

The present disclosure relates to one or more agents, therapies, treatments, and methods of use of the agents and/or therapies and/or treatments that inhibit the production or functionality of one or more checkpoint molecules. Non-limiting examples of checkpoint molecules include checkpoint proteins (CPPs) and phosphatidylserine (PS). Some embodiments of the present disclosure relate to methods for making a complex between at least one particle of an agent and at least one target cell of a subject where the complex decreases the production or functionality of one or more CPPs and/or PS. Embodiments of the present disclosure can be used as a therapy or a treatment for a patient.

In some embodiments of the present disclosure, a condition will or is likely to cause the subject's immune system to upregulate the expression of one or more checkpoint molecules.

In some embodiments of the present disclosure, the condition may be an acute or chronic infection of one the following non-limiting examples: a viral infection, a bacterial infection, a mycoplasma infection, a plasmodium infection, a amoeba infection, a fungus infection, a parasite infection, or combinations thereof.

In some embodiments of the present disclosure, the condition may be a cancer of one the following non-limiting examples: lung cancer, small cell lung cancer, non-small cell lung cancer, large cell lung cancer, renal cancer, colorectal cancer, bile duct cancer, penile cancer, melanoma cancer, non-melanoma skin cancer, cervical cancer, endometrial cancer, pancreatic cancer, breast cancer, oral cancer, brain cancer, glioma, astrocytoma, neuroblastoma, prostate cancer, adrenal cancer, anal cancer, thyroid cancer, bone cancer, osteosarcoma, soft tissue sarcoma, uterine cancer, fallopian tube cancer, spinal cancer, testicular cancer, head and neck cancer, ovarian cancer, vaginal cancer, vulvar cancer, stomach cancer, squamous cell cancer, sinus cancer, throat cancer, oral cancer, ocular cancer, liver cancer, intestinal cancer, gall bladder cancer, cancers of the lymph node, esophageal cancer or combinations thereof.

In some embodiments of the present disclosure, the agent can be administered to the subject by an intravenous route, an intramuscular route, an intraperitoneal route, an intrathecal route, an intravesical route, a topical route, an intranasal route, a transmucosal route, a pulmonary route, or combinations thereof.

In some embodiments of the present disclosure, the agent can be administered to the subject by pipetting a dose of the agent into an in vitro cell culture; perfusing or immersing an ex vivo cell or tissue preparation with a solution that comprises the agent; mixing a biological fluid sample with a solution or substrate that comprises the agent, or combinations thereof.

Some embodiments of the present disclosure relate to an agent that can be administered to a subject with the condition. When a therapeutically effective amount of the agent is administered to the subject, the subject may change production and/or functionality of one or more immune-system molecules. For example, the subject may increase or decrease production and/or functionality of one or more immune-system signaling molecules and/or one or more immune-system effector molecules by changing the production of one or more sequences of DNA, one or more sequences of RNA and/or one or more proteins and/or one or more regulatory molecules that regulate the subject's levels and/or functionality of the signaling immune-system molecules and/or the immune-system effector molecules.

In some embodiments of the present disclosure, the subject may respond to receiving a therapeutic amount of the agent by changing production and/or functionality of one or more intermediary molecules by changing production of one or more DNA sequences, one or more RNA sequences, and/or one or more proteins that regulate the levels and/or functionality of the one or more intermediary molecules. The one or more intermediary molecules regulate the subject's levels and/or functionality of the one or more immune-system signaling molecules and/or the one or more immune-system effector molecules.

In some embodiments of the present disclosure, administering a therapeutic amount of the agent to a subject decreases the production, functionality or both of one or more checkpoint molecules. The agent can reduce the production and/or activity of the one or more checkpoint molecules by increasing one or more of: synthesis of one or more nucleotides, nucleosides, sequences or genes that are related to decreasing the amounts or functionality of one or more checkpoint molecules; transcription of RNA that is related to decreasing the amount or functionality of one or more checkpoint molecules; translation of one or more amino acids or amino acid sequences that are related to decreased amounts or functionality of one or more checkpoint molecules; or combinations thereof. Examples of checkpoint molecules that the agent can decrease the production or functionality of include, but are not limited to: inhibitory checkpoint biomolecules, such as inhibitory CPPs and/or inhibitory checkpoint lipids. Non-limiting examples of inhibitory CPPs include: cytotoxic T-lymphocyte associated protein 4 (CTLA-4), programmed cell death protein 1 (PD-1), programmed death ligand 1 (PD-L1), programmed death ligand 2 (PD-L2), indoleamine 2,3-dioxygenase 1 (IDO1) or combinations thereof. A non-limiting example of an inhibitory checkpoint lipid is the phospholipid PS.

In some embodiments of the present disclosure, administering a therapeutic amount of the agent to a subject increases the production, functionality or both of one or more regulatory molecules that inhibits the production or functionality of one or more checkpoint molecules. The one or more regulatory molecules can be a sequence of DNA, RNA or amino acids that inhibits the production and/or functionality of one or more checkpoint molecules after administration of the agent. The agent can increase the production and/or functionality of the one or more regulatory molecules by increasing one or more of: synthesis of one or more nucleotides, nucleosides, sequences or genes that are related to causing increased amounts or functionality of the one or more regulatory molecules; transcription of RNA that is related to increased amounts or functionality of the one or more regulatory molecules; or translation of one or more amino acids or amino acid sequences that cause increased amounts or functionality of the one or more regulatory molecules.

In some embodiments of the present disclosure, the agent can be: a vector used for gene therapy; one or more selected nucleotides, a sequence of nucleotides, one or more nucleosides, a sequence of nucleosides, a DNA complex, one or more amino acids, a sequence of amino acids, a live microorganism, an attenuated microorganism, a dead microorganism, a recombinant virus, a non-recombinant virus, or combinations thereof.

In some embodiments of the present disclosure, the agent is a gene vector used for gene therapy. The gene therapy is useful for increasing the production of one or more checkpoint-molecule antagonists or for increasing the production of one or more regulatory molecules that inhibit the production or functionality of one or more checkpoint molecules. In some embodiments of the present disclosure, the vector can contain one or more genes that cause increased expression of one or more antagonists or regulatory molecules that inhibit the production and/or functionality of CTLA-4, PD-1, PD-L1, PD-L2, IDOL PS, other inhibitory checkpoint biomolecules or combinations thereof. In some embodiments of the present disclosure, the vector can contain one or more genes that cause a decreased expression of CTLA-4, PD-1, PD-L1, PD-L2, IDO1, PS, or combinations thereof.

In some embodiments of the present disclosure, the gene vector is a virus that can be within one or more of the following genus: flavivirus, influenza, enterovirus, rotavirus, rubellavirus, rubivirus, morbillivirus, orthopoxvirus, varicellovirus, dependoparvovirus, alphabaculovirus, betabaculovirus, deltabaculovirus, gammabaculovirus, mastadenovirus, simplexvirus, varicellovirus, cytomegalovirus, or combinations thereof. In some embodiments of the present disclosure the virus is an attenuated virus.

The embodiments of the present disclosure also relate to administering a therapeutically effective amount of the agent. The therapeutically effective amount of the agent will not substantially increase any deleterious conditions within the subject. For example, the therapeutically effective amount will not cause cytokinesis, hypercytokinemia, or any other uncontrolled, or partially controlled, upregulation of the subject's immune system. In some embodiments of the present disclosure, the therapeutically effective amount of the agent that is administered to a patient is between about 10 and about 1×10¹⁶ TCID₅₀/kg (50% tissue culture infective dose per kilogram of the patient's body weight). In some embodiments of the present disclosure the therapeutically effective amount of the agent that is administered to the patient is about 1×10¹³ TCID₅₀/kg. In some embodiments of the present disclosure, the therapeutically effective amount of the agent that is administered to a patient is measured in TPC/kg (total particle count of the agent per kilogram of the patient's body weight). In some embodiments the therapeutically effective amount of the agent is between about 10 and about 1×10¹⁶ TCP/kg.

Some embodiments of the present disclosure relate to a method for making an agent/target cell complex within a subject. The method comprises a step of administering a therapeutically effective amount of the agent to the subject. The complex comprises at least one particle of an agent and one or more target cells. When the complex is formed, it affects a change in metabolism of the one or more target cells so that results in the subject down regulating the production and/or functionality of one or more checkpoint molecules.

Some examples of a target cell that can form the agent/target cell complex include, but are not limited to: an adrenal gland cell; a B cell; a bile duct cell; a chondrocyte; a cochlear cell; a corneal cell; an endocardium cell; an endometrial cell; an endothelial cell; an epithelial cell; an eosinophil; a fibroblast; a hair follicle cell; a hepatocyte; a lymph node cell; a macrophage; a mucosal cell; a myocyte; a neuron; a glomeruli cell; an optic nerve cell; an osteoblast; an ovarian tissue cell; a pancreatic islet beta cell; a pericardium cell; a platelet; a red blood cell (RBC); a retinal cell; a scleral cell; a Schwann cell; a T cell; a testicular tissue cell; a thyroid gland cell; a uveal cell; or combinations thereof.

Further examples of a target cell that can form the agent/target cell complex include, but are not limited to: a lung cancer cell, a small cell lung cancer cell, a non-small cell lung cancer cell, a large cell lung cancer cell, a renal cancer cell, a colorectal cancer cell, a penile cancer cell, a bile duct cancer cell, a melanoma cancer cell, a non-melanoma skin cancer cell, a pancreatic cancer cell, a breast cancer cell, a cervical cancer cell, an endometrial cancer cell, a fallopian tube cancer cell, a throat cancer cell, an oral cancer cell, a prostate cancer cell, a brain cancer cell, a glioma cell, an astrocytoma cancer cell, a neuroblastoma cancer cell, an adrenal cancer cell, an anal cancer cell, a thyroid cancer cell, a bone cancer cell, an osteosarcoma sarcoma cell, a soft tissue sarcoma cell, a uterine cancer cell, a spinal cancer cell, a testicular cancer cell, a head and neck cancer cell, an ovarian cancer cell, a vaginal cancer cell, a vulvar cancer cell, a stomach cancer cell, a squamous cell cancer cell, a sinus cancer cell, a throat cancer cell, an ocular cancer cell, a liver cancer cell, an intestinal cancer cell, a lymph node cancer cell, a gall bladder cancer cell, an esophageal cancer cell and combinations thereof.

Some embodiments of the present disclosure relate to a therapy that can be administered to a subject with the condition. The therapy comprises a step of administering to the subject a therapeutically effective amount of an agent that will decrease production or activity of one or more regulatory molecules and/or one or more checkpoint molecules. When the therapy is administered to a patient, the therapy will decrease the in vivo production and/or functionality of one or more regulatory molecules and/or one or more checkpoint molecules. The decreased production and/or functionality of the checkpoint molecules may reduce or remove the deleterious effects of the condition upon the patient.

Some embodiments of the present disclosure relate to a method of treating a condition where the method comprises a step of administering to the subject a therapeutically effective amount of an agent that will decrease production or activity of one or more regulatory molecules and/or one or more checkpoint molecules.

Some embodiments of the present disclosure relate to therapies, treatments, and methods of use of more than one agent for inhibiting the production and/or functionality of more than one checkpoint molecule. For example, a therapy, treatment and method may comprise the use of: a first agent that can be used to inhibit the production and/or functionality of a first checkpoint molecule: a second agent can be used to inhibit the production and/or functionality of a checkpoint molecule; a third agent can be used to inhibit the production and/or functionality of a third checkpoint molecule. In some embodiments of the present disclosure there may be three, four, five or more agents used to inhibit the production and/or functionality of three or more checkpoint molecules.

Some embodiments of the present disclosure relate to therapies, treatments, and methods of use of one, two or three of the first agent, the second agent, the third agent and another agent and/or further agents that inhibit the production and/or functionality of one or more CPPs and/or PS. The first agent, the second agent, the third agent and the another agent and/or further agents can each increase the production and/or functionality of an antagonist for each of the agents' respective target checkpoint molecule.

In some alternative embodiments of the present disclosure, the first agent, the second agent, the third agent and the another agent and/or further agents can each increase the production and/or functionality of a regulatory molecule that, directly or indirectly, inhibits the production and/or functionality of each of the agents' respective target checkpoint molecule.

### Example 1

A male patient of Caucasian ancestry in his early 40's presented with stage IVB metastatic colorectal cancer with metastatic lesions detected in the liver, lung, and lymph nodes. The patient's previous treatment history included surgery and radiation therapy for the primary lesion, FOLFOX/bevacizumab, and FOLFIRI/cetuximab. The patient's condition, which did not progress after each of the previous treatments. The patient was then treated with four cycles of ipilimumab, an anti CTLA-4 drug, pembrolizumab, an anti PD-1 drug, and atezolizumab, an anti PD-L1 drug. After therapy the patient demonstrated no evidence of any cancer lesions and has been in complete remission for at least two years.

1. A method of making an agent/target cell complex, the method comprising a step of administering a therapeutically effective amount of an agent to a subject, wherein the agent/target cell complex increases the subject's endogenous production and/or a functionality of an antagonist of a checkpoint molecule.

2. The method of claim 1, wherein the antagonist is an inhibitory checkpoint antagonist.

3. The method of claim 1, wherein the antagonist is a checkpoint protein antagonist and/or a phosphatidylserine antagonist.

4. The method of claim 2, wherein the antagonist is a regulatory molecule.

5. The method of claim 1, wherein the checkpoint molecule is at least one of cytotoxic T-lymphocyte associated protein 4 (CTLA-4), programmed cell death protein 1 (PD-1), programmed death ligand 1 (PD-L1), programmed death ligand 2 (PD-L2), indoleamine 2,3-dioxygenase 1 (IDO1) and phosphatidylserine.

6. The method of claim 1, wherein the agent is at least one of a gene vector used for gene therapy, one or more selected nucleotides, a sequence of nucleotides, one or more nucleosides, a sequence of nucleosides, a DNA complex, one or more amino acids, a sequence of amino acids, a live microorganism, an attenuated microorganism, a dead microorganism, a recombinant virus and a non-recombinant virus.

7. The method of claim 1, wherein the agent/target cell complex comprises a target cell that is at least one of an adrenal gland cell; a B cell; a bile duct cell; a chondrocyte; a cochlear cell; a corneal cell; an endocardium cell; an endometrial cell; an endothelial cell; an epithelial cell; an eosinophil; a fibroblast; a hair follicle cell; a hepatocyte; a lymph node cell; a macrophage; a mucosal cell; a myocyte; a neuron; a glomeruli cell; an optic nerve cell; an osteoblast; an ovarian tissue cell; a pancreatic islet beta cell; a pericardium cell; a platelet; a red blood cell (RBC); a retinal cell; a scleral cell; a Schwann cell; a T cell; a testicular tissue cell; a thyroid gland cell; and a uveal cell.

8. The method of claim 1, wherein the agent/target cell complex comprises a target cell that is at least one of a lung cancer cell, a small cell lung cancer cell, a non-small cell lung cancer cell, a large cell lung cancer cell, a renal cancer cell, a colorectal cancer cell, a penile cancer cell, a bile duct cancer cell, a melanoma cancer cell, a non-melanoma skin cancer cell, a pancreatic cancer cell, a breast cancer cell, a cervical cancer cell, an endometrial cancer cell, a fallopian tube cancer cell, a throat cancer cell, an oral cancer cell, a prostate cancer cell, a brain cancer cell, a glioma cell, an astrocytoma cancer cell, a neuroblastoma cancer cell, an adrenal cancer cell, an anal cancer cell, a thyroid cancer cell, a bone cancer cell, an osteosarcoma sarcoma cell, a soft tissue sarcoma cell, a uterine cancer cell, a spinal cancer cell, a testicular cancer cell, a head and neck cancer cell, an ovarian cancer cell, a vaginal cancer cell, a vulvar cancer cell, a stomach cancer cell, a squamous cell cancer cell, a sinus cancer cell, a throat cancer cell, an ocular cancer cell, a liver cancer cell, an intestinal cancer cell, a lymph node cancer cell, a gall bladder cancer cell, and an esophageal cancer cell.

9. The method of claim 1, further comprising a step of administering a second agent that decreases a production and/or a functionality of a second checkpoint molecule.

10. The method of claim 9, wherein the second agent second increases a production and/or a functionality of a regulatory molecule that decreases the production and/or the functionality of the second checkpoint molecule.

11. The method of claim 9, further comprising a step of administering a third agent that decreases a production and/or a functionality of a third checkpoint molecule.

12. The method of claim 11, wherein the third agent increases the production and/or functionality of a regulatory molecule that decreases the production and/or the functionality of the third checkpoint molecule.

13. The method of claim 11, further comprising a step of administering a fourth agent that decreases a production and/or a functionality of a fourth checkpoint molecule.

14. The method of claim 13, wherein the fourth agent increases the production and/or functionality of a regulatory molecule that decreases the production and/or the functionality of the fourth checkpoint molecule.

15. The method of claim 13, further comprising a step of administering a fifth agent that decreases a production and/or a functionality of a fifth checkpoint molecule.

16. The method of claim 15, wherein the fifth agent increases the production and/or functionality of a regulatory molecule that decreases the production and/or the functionality of the fifth checkpoint molecule.

17. The method of claim 15, further comprising a step of administering a sixth agent that decreases a production and/or a functionality of a sixth checkpoint molecule.

18. The method of claim 17, wherein the sixth agent increases the production and/or functionality of a regulatory molecule that decreases the production and/or the functionality of the sixth checkpoint molecule.

19. A pharmaceutical composition comprising:
a. an agent that decreases a production and/or a functionality of one or more checkpoint molecules and/or the agent increases a production and/or a functionality of one or more regulatory molecules that decrease a production and/or a functionality of the one or more checkpoint molecules;
b. a pharmaceutically acceptable carrier; and/or
c. an excipient.

20. The pharmaceutical composition according to claim 19, wherein the one or more checkpoint molecules is an inhibitory checkpoint molecule.

21. The pharmaceutical composition according to claim 19, wherein the one or more checkpoint molecules is at least one of cytotoxic T-lymphocyte associated protein 4 (CTLA-4), programmed cell death protein 1 (PD-1), programmed death ligand 1 (PD-L1), programmed death ligand 2 (PD-L2), indoleamine 2,3-dioxygenase 1 (IDO1) and phosphatidylserine.

22. The pharmaceutical composition of claim 19, further comprising a second agent that decreases a production and/or a functionality of a second checkpoint molecule.

23. The pharmaceutical composition of claim 22, wherein the second agent increases a production and/or a functionality of a second regulatory molecule that decreases the production and/or the functionality of the second checkpoint molecule.

24. The pharmaceutical composition of claim 22, further comprising a third agent that decreases a production and/or a functionality of a third checkpoint molecule.

25. The pharmaceutical composition of claim 24, wherein the third agent increases a production and/or a functionality of a regulatory molecule that decreases the production and/or the functionality of the third checkpoint molecule.

26. The pharmaceutical composition of claim 24, further comprising a fourth agent that decreases a production and/or a functionality of a fourth checkpoint molecule.

27. The pharmaceutical composition of claim 26, wherein the fourth agent increases a production and/or a functionality of a regulatory molecule that decreases the production and/or the functionality of the fourth checkpoint molecule.

28. The pharmaceutical composition of claim 26, further comprising a fifth agent that decreases a production and/or a functionality of a fifth checkpoint molecule.

29. The pharmaceutical composition of claim 28, wherein the fifth agent increases a production and/or a functionality of a regulatory molecule that decreases the production and/or the functionality of the fifth checkpoint molecule.

30. The pharmaceutical composition of claim 28, further comprising a sixth agent that decreases a production and/or a functionality of a sixth checkpoint molecule.

31. The pharmaceutical composition of claim 30, wherein the sixth agent increases a production and/or a functionality of a regulatory molecule that decreases the production and/or the functionality of the sixth checkpoint molecule.

32. The pharmaceutical composition according to claim 19, wherein the pharmaceutical composition is in a solid form or a liquid form.

33. A method of treating an infection, the method comprising a step of administering to a subject a therapeutically effective amount of an agent that decreases a production and/or a functionality of three or more checkpoint molecules.

34. The method according to claim 33, wherein the infection is at least one of a chronic viral infection, a chronic bacterial infection, a chronic mycoplasma infection, a chronic plasmodium infection, a chronic amoeba infection, a chronic fungus infection, and a chronic parasite infection.

35. The method according to claim 33, wherein the agent decreases the production and/or the functionality of four or more checkpoint molecules.

36. The method according to claim 33, wherein the agent decreases the production and/or the functionality of five or more checkpoint molecules.

37. The method according to claim 33, wherein the agent decreases the production and/or the functionality of six or more checkpoint molecules.

38. The method according to claim 33, wherein the three or more checkpoint molecules are inhibitory checkpoint molecules.

39. The method according to claim 33, wherein at least one of the three or more checkpoint molecules are a checkpoint protein.

40. The method according to claim 33, wherein at least one of the three or more checkpoint molecules are a checkpoint lipid.

41. A method of treating a cancer, the method comprising a step of administering to a subject a therapeutically effective amount of an agent that decreases a production and/or a functionality of three or more checkpoint molecules.

42. The method according to claim 41, wherein the cancer is at least one of lung cancer, small cell lung cancer, non-small cell lung cancer, large cell lung cancer, renal cancer, colorectal cancer, bile duct cancer, penile cancer, melanoma cancer, non-melanoma skin cancer, cervical cancer, endometrial cancer, pancreatic cancer, breast cancer, oral cancer, brain cancer, glioma, astrocytoma, neuroblastoma, prostate cancer, adrenal cancer, anal cancer, thyroid cancer, bone cancer, osteosarcoma, soft tissue sarcoma, uterine cancer, fallopian tube cancer, spinal cancer, testicular cancer, head and neck cancer, ovarian cancer, vaginal cancer, vulvar cancer, stomach cancer, squamous cell cancer, sinus cancer, throat cancer, oral cancer, ocular cancer, liver cancer, intestinal cancer, gall bladder cancer, cancers of the lymph node, and esophageal cancer.

43. The method of treating cancer of claim 41, wherein the agent decreases the production and/or functionality of four or more checkpoint molecules.

44. The method of treating cancer of claim 41, wherein the agent decreases the production and/or functionality of five or more checkpoint molecules.

45. The method of treating cancer of claim 41, wherein the agent decreases the production and/or functionality of six or more checkpoint molecules.

46. The method according to claim 41, wherein the step of administering occurs by at least one of an intravenous route, an intramuscular route, an intraperitoneal route, an intrathecal route, an intravesical route, a topical route, an intranasal route, a transmucosal route, and a pulmonary route.

47. The method according to claim 41, wherein each of the three or more checkpoint molecules is cytotoxic T-lymphocyte associated protein 4 (CTLA-4), programmed cell death protein 1 (PD-1), programmed death ligand 1 (PD-L1), programmed death ligand 2 (PD-L2), indoleamine 2,3-dioxygenase 1 (IDO1) and phosphatidylserine and combinations thereof.

48. The method according to claim 41, wherein the agent increases a production and/or a functionality of a regulatory molecule that decreases the production of or the functionality of at least one of the three or more checkpoint molecules.

49. The method according to claim 48, wherein the regulatory molecule is a sequence of DNA and/or a sequence of RNA that decreases production of at least one of the three or more checkpoint molecules.

50. The method according to claim 41, wherein the agent is a vector containing a gene or genes for decreasing the levels of at least one of CTLA-4, PD-1, PD-L1, PD-L2, IDO1 and PS.

51. The method according to claim 41, wherein the therapeutically effective amount is between about 10 to about 1×10¹⁶ TCID₅₀/kg of the patient's body weight.

52. The method according to claim 41, wherein the therapeutically effective amount is between about 10 to about 1×10¹⁶ total particles of the agent.
